(19) United States
(12) Patent Application Publication
(10) Pub. No.: US 2011/0144450 A1
(43) Pub. Date: Jun. 16, 2011

(54) Tissue retractor with movable blades and articulating wrist joint

(76) Inventors: Anthony Paolitto, Montreal (CA); Jonathan Paquette, Blainville (CA); Rene Sylvestre, Laval (CA); Valerio Valentini, Montreal (CA)

(21) Appl. No.: 12/926,018

(22) Filed: Oct. 19, 2010

Related U.S. Application Data
(60) Provisional application No. 61/272,668, filed on Oct. 19, 2009.

(51) Int. Cl. A61B 1/32

(52) U.S. Cl. 600/224

(57) ABSTRACT

A surgical retractor for retracting a body tissue comprising a plurality of cooperating tissue-retracting blades connected to a retractor housing through a linkage arrangement. The tissue-retracting blades are movable between a closed-blade configuration wherein said blades are in proximity to one another and an open-blade configuration wherein said blades are in a spaced apart spatial relationship. The blade spatial relationship being variably selectable by the degree of actuation input applied to a first actuator that is coupled to both the retractor housing and the linkage arrangement. The tissue-retracting blades are also movable, together as a blade assembly in a selected blade spatial relationship, when a second actuation input is applied to a second actuator to articulate a pivoting wrist joint configured in the retractor housing. Actuating the wrist joint through the second actuator allows the surgeon to vary the orientation of the spaced apart blades relative to the housing through an angular displacement of the blades about a wrist pivot axis.

This application claims the benefits of U.S. Provisional Patent Application 61/272,668 filed Oct. 19, 2009.

### FIELD OF THE INVENTION

The present invention relates to the field of surgical instruments to retract a body tissue and more specifically, to cardiac tissue retractors that are adapted for use in heart surgery to retract a portion of a patient's heart, said retractors being configured with a plurality of movable tissue-retracting blades or fingers that are able to assume a variably selectable spatial relationship therebetween.

### BACKGROUND OF THE INVENTION

Current tissue retractors, especially in cardiac surgery, are typically of a fixed geometry. They are most commonly configured at the tissue-retracting end with either a “basket” type configuration made from fixed non-movable spaced apart wire frame members, or with an uninterrupted and shaped tissue contacting surface or blade that engages the cardiac or heart tissue to be retracted. These retractors are most typically employed to retract the cardiac tissue comprising the left atrium of the heart during a surgery on the mitral heart valve, or the cardiac tissue comprising the right atrium during a surgery on the tricuspid heart valve. During retraction of said atria by said known retractors, the latter are not adaptable or adjustable to suit the specific anatomy being retracted by selectively varying the geometry of the tissue retracting end or the spatial relationship of the spaced apart wire frame members. These known retractors are not provided with an actuation means or member to variably select the spatial relationship or relative position between tissue-retracting members by the degree of actuation input applied to the actuation means or actuator. Furthermore, these known retractors are not provided with a second actuation means or member that may also additionally vary the orientation of the tissue-retracting blades relative to the housing which said tissue-retracting blades are coupled to. Moreover, these known retractors are not provided said actuation means or actuators that may be actuated extracorporeally by the user when said tissue-retracting portion of said tissue retractor is located within the body or a cavity thereof so as to produce a distal movement of tissue-retracting blades by applying an actuation input to said actuators located proximally to said user. Tissue retractors with movable tissue retracting blades, whose spatial relationship may be adjusted or selected by remotely manipulating an actuator, are particularly advantageous for use in laparoscopic surgery or intercostal cardiac surgery when the tissue engaging or retracting blades are contained within a body or chest cavity and not easily or directly accessible to the surgeon or user during the surgical procedure, especially when the latter are engaged with a target anatomic tissue being retracted. With less-invasive laparoscopic or port access surgeries gaining in popularity, having a surgical retractor with laterally spreading blades actuated by a first actuator, and also being able, by actuating a second actuator, to variably select or modify the orientation of said laterally spreading blades relative to the housing to which they are coupled is advantageous in allowing the surgeon user to: i) vary the span of retraction between tissue-retracting blades, and ii) change the retraction orientation of said blades relative to the housing at a given span of retraction. Such instrument adjustability, while tissue-engaging blades remain in contact with a target tissue being retracted, provides the surgeon with improved surgical access, and facilitates the repositioning and reorientation of said tissue retractor during the different phases of the surgical procedure without having to greatly redo the surgical set up. Moreover, in laparoscopic or port access surgeries, said instrument adjustability allows the functional intra-corporeal end of the tissue retractor to be repositioned or reoriented within the body by extracorporeal manipulation of one or both actuators. As such, unlike known tissue retractors where the tissue engaging blade is in a fixed spatial relationship relative to its housing, this instrument adjustability alleviates the need to have to re-introduce the tissue retractor through a separate port or incision if the orientation of the tissue-engaging blades is not optimum relative to the target tissue.

### SUMMARY OF THE INVENTION

Thus, it is a first object of the present invention to provide a tissue retractor having a plurality of cooperating tissue-retracting or tissue-engaging blades or fingers connected to a retractor housing via a linkage assembly, said tissue-retracting blades being movable between a closed-blade configuration wherein said blades are in proximity to one another and an open-blade configuration wherein said blades are in a spaced apart spatial relationship, said blade spatial relationship being variably selectable by the degree of actuation applied to a first actuator for moving said blades relative to each other to change their relative position, said tissue-retracting blades also being pivotable, collectively as a blade assembly in a selected blade spatial relationship, about a pivot axis provided by a pivoting wrist joint configured in said retractor housing when a second actuation input is applied to a second actuator to articulate said pivoting wrist joint and to vary the orientation of the spaced apart blades relative to said housing.

It is a further object of the present invention to provide a cardiac tissue retractor comprised of a plurality of adaptable tissue-retracting or tissue-engaging blades coupled to a generally elongate retractor housing, said blades configured and sized to retract a cardiac tissue of the patient's heart, said plurality of tissue-retracting blades being adjustable or movable in position relative to each other by the actuation of a first actuator between a blade-closed configuration whereby said blades are in proximity to each other and a blade-open configuration whereby said blades are in a spaced apart spatial relationship so that, in use, the cardiac tissue retractor may be customized or tailored to suit the specific anatomy of the patient or the specific geometry of a surgical incision by variably selecting a desired spatial relationship of the said plurality of blades, said cardiac tissue retractor being further provided with a second actuator to selectively vary the orientation of the tissue-retracting blades relative to a tissue retractor housing, in their said desired blade spatial relationship, said housing being configured to house said first and second actuators.

It is a further object of the present invention to provide a tissue retractor comprising an elongated housing having a longitudinal axis and a plurality of movable tissue-retracting blades coupled to said housing, whereby, in use, when said housing is inserted in a surgical access port or into a surgical incision, said plurality of blades are moveable intracorporeally relative to each other to engage and retract a target anatomic tissue, the position of said plurality of blades relative to each other, and the blade angular orientation of said plurality of blades relative to said housing longitudinal axis may be varied through the extracorporeal actuation of a first and a second actuator, respectively.

These and other objects of the present invention will become apparent from the description of the present invention and its preferred embodiments which follows.

### BRIEF DESCRIPTION OF THE DRAWINGS

For better understanding of the present invention and to show more clearly how it may be carried into effect, reference will now be made by way of illustration and not of limitation to the accompanying drawings, which show a tissue retractor apparatus according to preferred embodiments of the present invention, and in which:

FIG. 1 is a perspective view of a cardiac tissue retractor mounted to a chest retractor 99 and comprising a plurality of movable tissue-retracting blades retracting a left atrium tissue, according to a preferred embodiment of the present invention;

FIG. 2 is a perspective view of the cardiac tissue retractor illustrated in FIG. 1, with the linkage assembly 30 decoupled from its retractor housing and with the tissue-retracting blades 40 in a closed-blade configuration to facilitate their introduction through an intercostal access port IAP into the patient's thoracic cavity;

FIG. 3 is a perspective view of the tissue retractor 1 with the linkage assembly 30 coupled to the retractor housing 20 at wrist joint 21 and with tissue-retracting blades in the open-blade configuration;

FIG. 4 is a perspective view of the tissue retractor 1 illustrating tissue-retracting blades 40 and linkage assembly 30 disengaged from retractor housing 20, actuation cable 22 and obturator 23 that can be inserted into a central passageway in retractor housing 20;

FIG. 5A is a perspective view of tissue retractor 1 with actuation cable 22 inserted in passageway of retractor housing, said cable 22 engaged with actuator knob 10 in a first position 11 relative to retractor housing 20, said cable extending outwardly from first housing end 25 and engaged with socket 31 in linkage assembly 30 prior to retracting cable 22 within retractor housing as actuator knob 10 is moved to second position 12 relative to housing 20;

FIG. 5B is a close up view illustrating the socket 31 in linkage assembly 30 and the ball end 221 in cable 22 extending from first housing end 25 of retractor housing 20;

FIG. 6A is a top view of tissue retractor 1 illustrating cable 22 extending outwardly from housing end 25, said cable 22 engaged with linkage assembly 30 though cable ball end 221 and socket 31, cable fitting 222 engaged in housing slot 24, and actuator knob 10 in first position 11 relative to housing 20, prior to linkage assembly 30 engaging housing end 25 adjacent wrist joint 21, and tissue-retracting blades 40 in blade-closed configuration 91;

FIG. 6B is a top view of tissue retractor 1 illustrating cable 22 retracted within housing 20 with actuator 10 moved to a second position 12, linkage assembly 30 engaged with housing 20 adjacent wrist joint 21, and tissue-retracting blades 40 in blade-closed configuration 91;

FIG. 6C is a top view of tissue retractor 1 illustrating linkage assembly 30 engaged with housing 20 adjacent wrist joint 21, cable 22 retracted within housing 20 and actuator 10 having been moved to second position 12 and subsequently actuated to move tissue-engaging blades 40 in a blade-open configuration 92;

FIG. 6D is a top view of tissue retractor 1 illustrating linkage assembly 30 engaged with housing 20 adjacent wrist joint 21, said linkage assembly 30 pivoted to one side of retractor housing 20, said pivoting allowed by flexible cable 22; tissue-engaging blades 40 in a blade-open configuration 92;

FIGS. 7A to 7C illustrates the range of movement of linkage assembly 30 and blades 40 relative to housing longitudinal axis 29, said movement resulting in a variable orientation of blades 40 relative to housing 20, said orientation defined by angle (−) that is achieved when second actuator 50 is actuated and wrist joint 21 is articulated;

FIGS. 8A to 8D illustrate cross-sectional views through a first embodiment of tissue retractor 1 according to the present invention;

FIGS. 9A to 9C illustrate cross-sectional views through the wrist joint 21 of tissue retractor 1;

FIGS. 10A to 10C illustrate cross-sectional views through a second embodiment of tissue retractor 2 according to the present invention;

FIGS. 11A to 11C illustrate the geometric relationship between planes and axes used to define the cardiac tissue retractor 1 according to the present invention;

FIGS. 12 to 12H illustrate the variety of different coupling arrangements available at demountable coupling joint 282 between linkage mechanism 30 (and plurality of tissue-engaging blades 40 attached thereto) and housing 20, and the relationship of PLN-T to PLN-W in each of the different coupled positions.

### DETAILED DESCRIPTION OF THE INVENTION

The invention will be described in the context of a cardiac valve surgery performed on the mitral valve of the patient. It is understood that the concepts and principles of the invention may be applied to tissue retracting apparatus used to perform cardiac surgery on the other cardiac valves (i.e. pulmonary, tricuspid, and aortic), or even to other tissue retracting apparatus used for retracting a target anatomic tissue contained within an internal body cavity of a patient's body, without departing from the spirit of the invention.

The heart is contained within a patient's thorax or thoracic cavity, and is located beyond a structural ribcage. The heart includes a number of internal cavities through which blood flows and which are associated with a heart valve. Included in these internal cavities are the heart chambers (left atrium, right atrium, left ventricle, right ventricle). Each of the heart chambers is delimited by a number of chamber-defining walls and inner chamber partitions or septal walls. As well, each of the heart chambers is delimited by at least one cardiac valve to control passage of blood flow through the chamber in a synchronized manner with each heart beat. Apart from the heart chambers and included in these internal cavities are the passageways or regions within the cardiac anatomy which are immediately adjacent or associated with a heart valve. For instance, the aortic root located just downstream and above the aortic valve is one such cavity which surgeons routinely access when performing a surgical procedure on the aortic valve (or the ascending aorta and the sinuses of Valsalva). The different heart valves (aortic, mitral, tricuspid, or pulmonary) have at least one valve cusp that is displaced between a valve closed and valve open configuration to selectively restrict or allow passage of blood therethrough.

The patient's heart is comprised of different cardiac tissues including tissue of the aorta, tissue of the vena cavae, tissue of the pulmonary veins and arteries, tissue of the left and right atria, tissue of the left and right ventricles, tissue of the atrial septum, and tissue of the ventricular septum. For the purposes of this description of the invention, the term “cardiac tissue” will include all tissues of the heart that may need to be retracted in order to gain surgical or visual access to a target region or target anatomic tissue of the heart such as a cardiac valve, a heart chamber, a vascular conduit, etc.

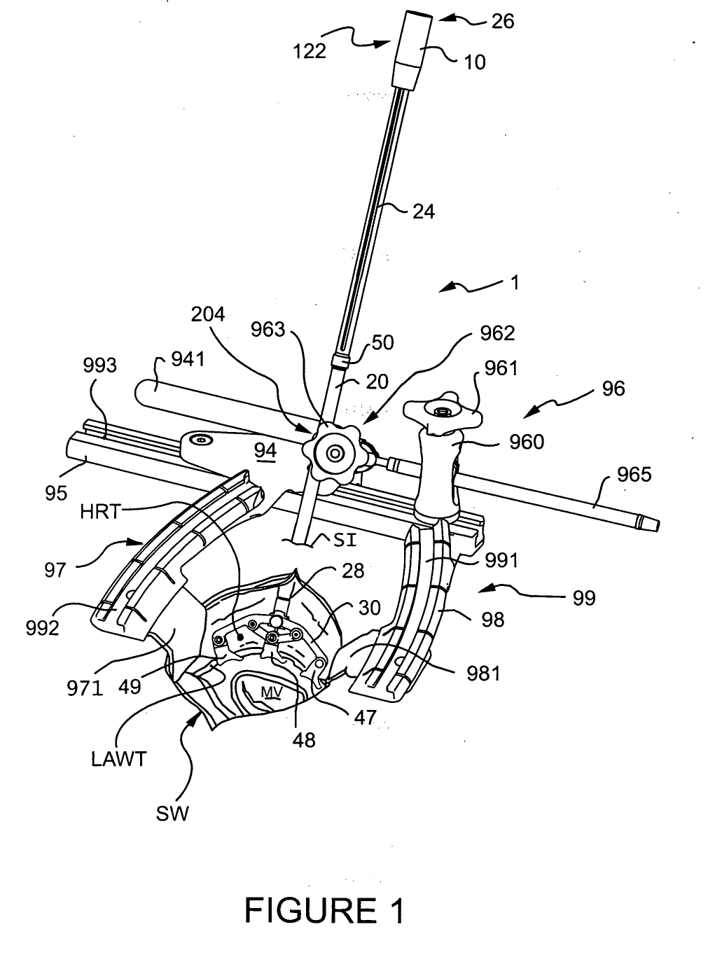

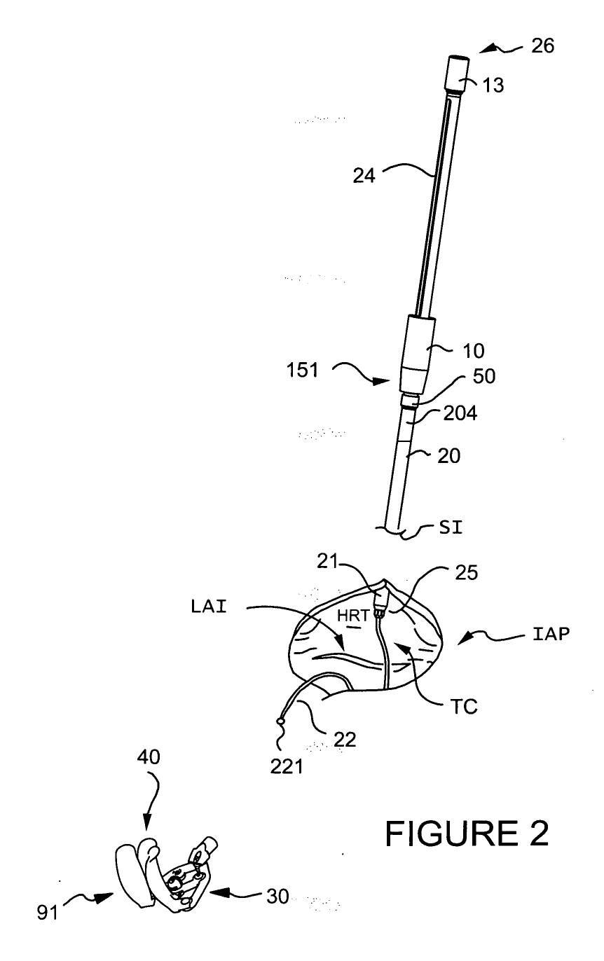

Referring to FIGS. 1 and 2, a patient's heart HRT is accessed via an intercostal access port IAP in a thoracic cavity TC. A left atriotomy incision or left atrial incision LAI in the left atrium of the heart HRT provides surgical and visual access to a mitral valve MV, and to the valve leaflets or cusps thereof.

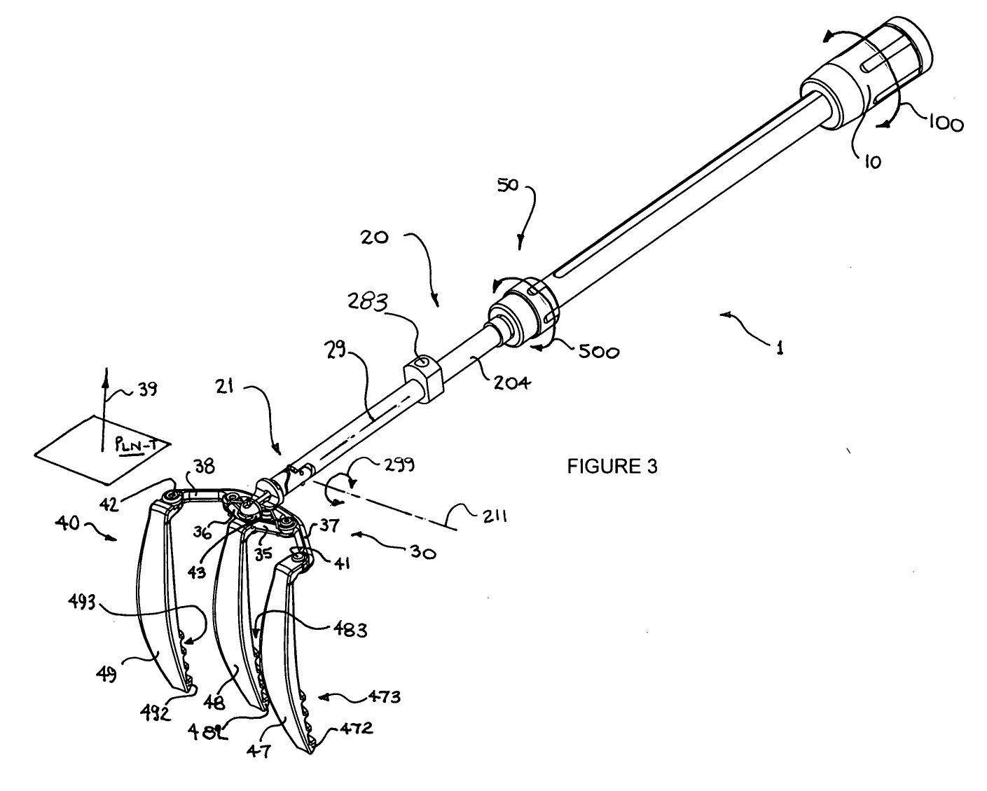

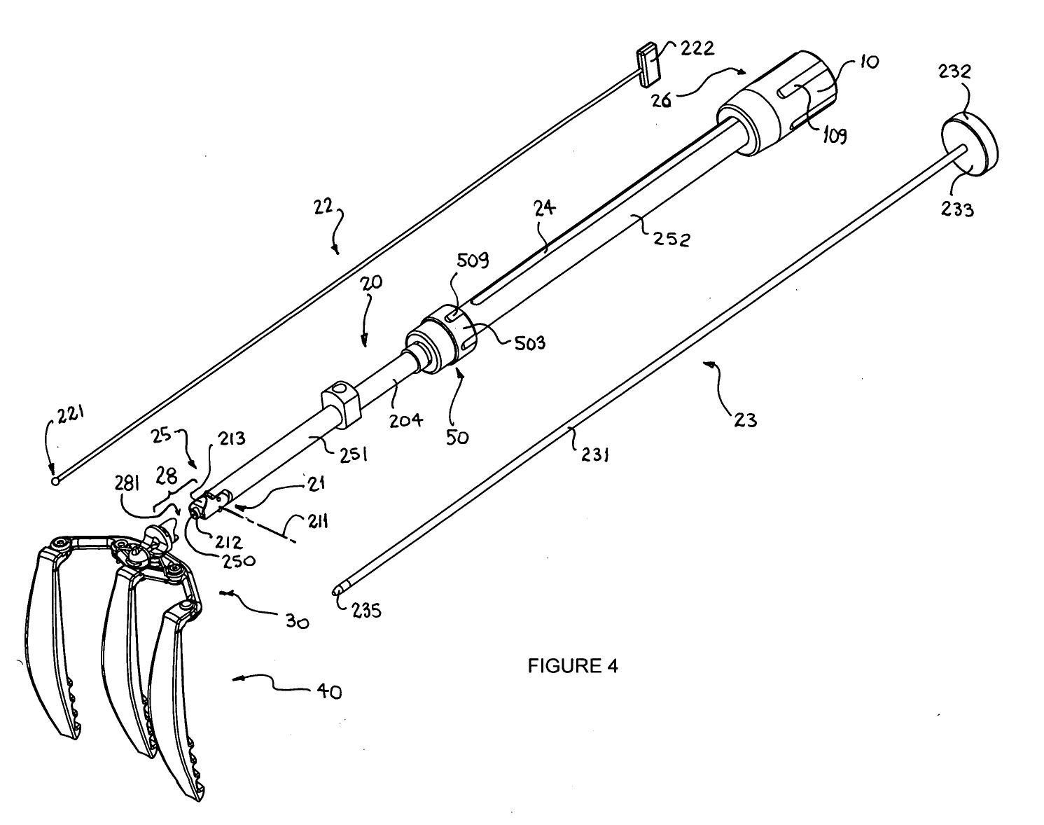

Referring to FIGS. 3 and 4, a first embodiment of a cardiac tissue retractor or apparatus 1 is comprised of a plurality of tissue-engaging or tissue-retracting blades 40, a linkage assembly or mechanism 30, a retractor housing 20, a first actuator 10 and a second actuator 50.

As illustrated in FIG. 1, cardiac tissue retractor 1 is preferably mounted to a substantially stable surgical platform, such as a chest retractor, or more specifically, an intercostal thoracic retractor 99 via an instrument positioning arm 96. Thoracic retractor 99 is comprised of a first, movable spreader arm 97 and a second, fixed spreader arm 98. Arms 97 and 98 are provided with blades 971, 988 respectively, said blades being configured and sized to spread apart two adjacent ribs of the patient's ribcage, in order to obtain surgical access to the underlying thoracic cavity TC and the patient's heart HRT located therewithin. Arm 97 moves relative to arm 98 along rack bar 95 when crank mechanism 94 is actuated by a rotation of pinion 941, and as such the relative lateral spacing between blades 971, 981, and the resulting surgical window SW may be controlled.

Instrument positioning arm 96 includes a first mechanical joint or clamp 960 which is provided with a key member or fitting (not shown) designed to slidingly engage or mate with perimeter rails 991, 992 or 993 of thoracic retractor 99. As such, joint 960 (and consequently arm 96) may be variably mounted anywhere along perimeter rails 991, 992 or 993. As well, mechanical joint 960 secures the position and orientation of arm member or rod 965 relative to thoracic retractor 99, and the position of mechanical joint 960 along anyone of said perimeter rails, when knob 961 is tightened. Instrument positioning arm 96 also includes a second mechanical joint or clamp 962 which is configured to engage with and clamp cardiac tissue retractor 1. Cardiac tissue retractor 1 is provided with a retractor-mounting-interface, or mounting seat 204 which advantageously allows said retractor 1 to be engaged within said clamp member 962. Clamp member 962 provides multiple motion degrees of freedom thus allowing the surgeon to vary the angular orientation between housing 20 and rod 965. Tightening clamp knob 963 results in securing said angular orientation. As such, through instrument positioning arm 96, the position and orientation of cardiac tissue retractor 1 may be secured in desired spatial relationship relative to thoracic retractor 99 (and also the patient's thorax which retractor 99 is engaged with) when clamp knobs 961, 963 are tightened. This allows the surgeon to impart the desired tissue retraction to a cardiac tissue and then secure this retraction load by clamping the cardiac tissue retractor 1 to thoracic retractor 99 in the optimum retracting position and orientation.

It is understood that cardiac tissue retractor 1 may alternatively be mounted to other types of surgical platforms via positioning arm 96 or even other types of instrument positioning arms. For instance, tissue retractor 1 may be mounted to a surgical table via a multi-jointed articulating surgical arm well known in the field of endoscopic surgery. For instance, tissue retractor 1 may be mounted to a sternotomy chest retractor configured with a perimeter rail 991, 992, or 993 via instrument positioning arm 96.

Referring to FIGS. 3 to 5B, plurality of cardiac tissue-engaging or cardiac tissue-retracting blades 40 includes three cooperating blades or fingers 47, 48, 49. As illustrated, said tissue-retracting blades are suitably configured and appropriately sized to engage with and retract a cardiac tissue, in this case, portion of the incised left atrium or left atrial wall tissue LWAT, thereby providing the surgeon with surgical access to the mitral valve MV (i.e. the target heart valve) via a left atrial incision LAI. Accordingly, terminal blade ends 472, 482, 492 are bent and configured with a hook-like geometry adapted to hook the LAWT and minimize slipping of said cardiac tissue relative to said blades 47, 48, 49 when a retracting load is applied to tissue retracting apparatus 1. As well, said terminal ends are also profiled to be blunt and atraumatic so as to not pierce through the cardiac tissue being retracted. In order to further enhance the friction or traction exerted by said blades on said cardiac tissue being retracted, said blades may also be preferably configured with a number of spaced-apart ridges 473, 483, 493 along the tissue-contacting surface of said blades. Preferably, blades 47, 48, 49 are sized with a blade length BL from 1.2 to 2.4 inches (30 to 60 mm), and a blade width BW from 0.275 to 0.470 inches (7 to 12 mm). Other sizes are also suitable, depending on the size of the patient's heart HRT and size of left atrium to be retracted, or other cardiac tissue being retracted.

Each of said tissue-engaging blades 47, 48, 49 is preferably pivotingly connected to movable linkage mechanism 30 at a separate blade mount location, interface, or blade-to-link or blade-to-linkage joint 41, 42, 43, respectively. As such, said blades may pivot and orient themselves relative to the cardiac tissue being retracted to assume a less traumatic blade orientation. This blade adaptability tends to provide substantially equal or equilibrated reaction loads being applied by each blade to the blade-contacted portion of body tissue being retracted. A PLN-T may be defined through said joints 41, 42, 43. A vector 39 is also used to define said PLN-T. When first actuator 10 is actuated, said joints move relative to one another within said plane PLN-T as tissue-retracting blades 47, 48, 49 move between said closed-blade 91 and said open-blade configuration 92. As illustrated, blades 47, 48, 49 extend away from PLN-T in a substantially perpendicular direction. Alternatively, blades may also be configured to extend away from PLN-T with an angular orientation whilst joints 41, 42, 43 are still movable within PLN-T.

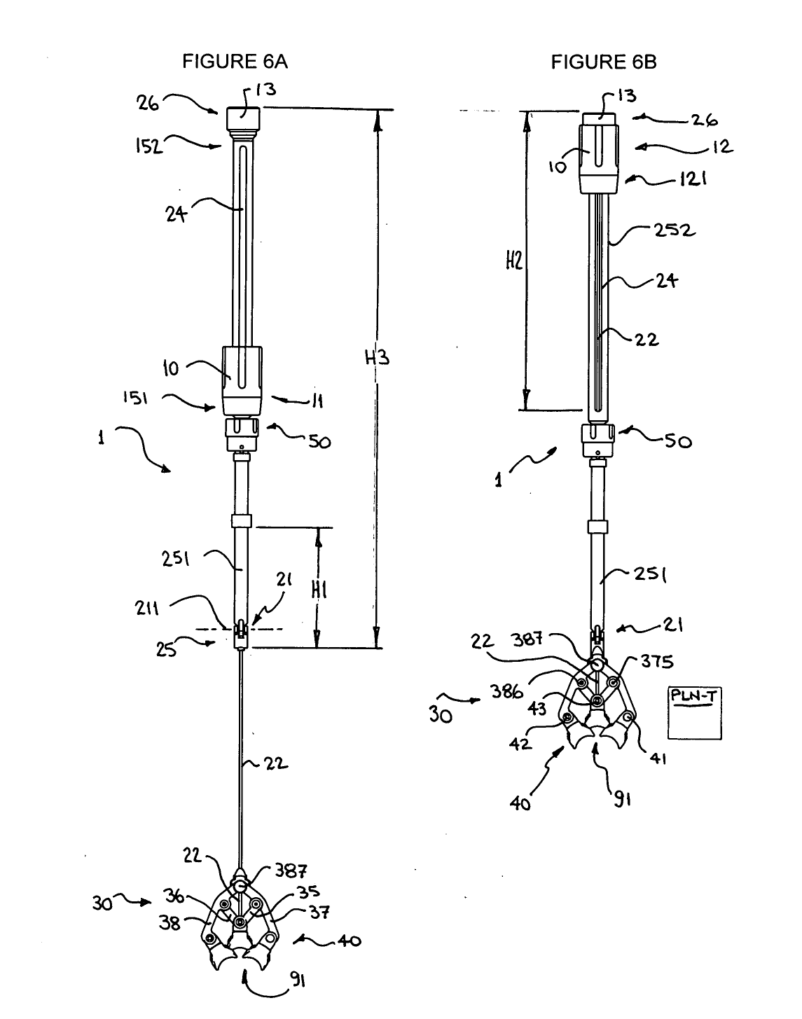

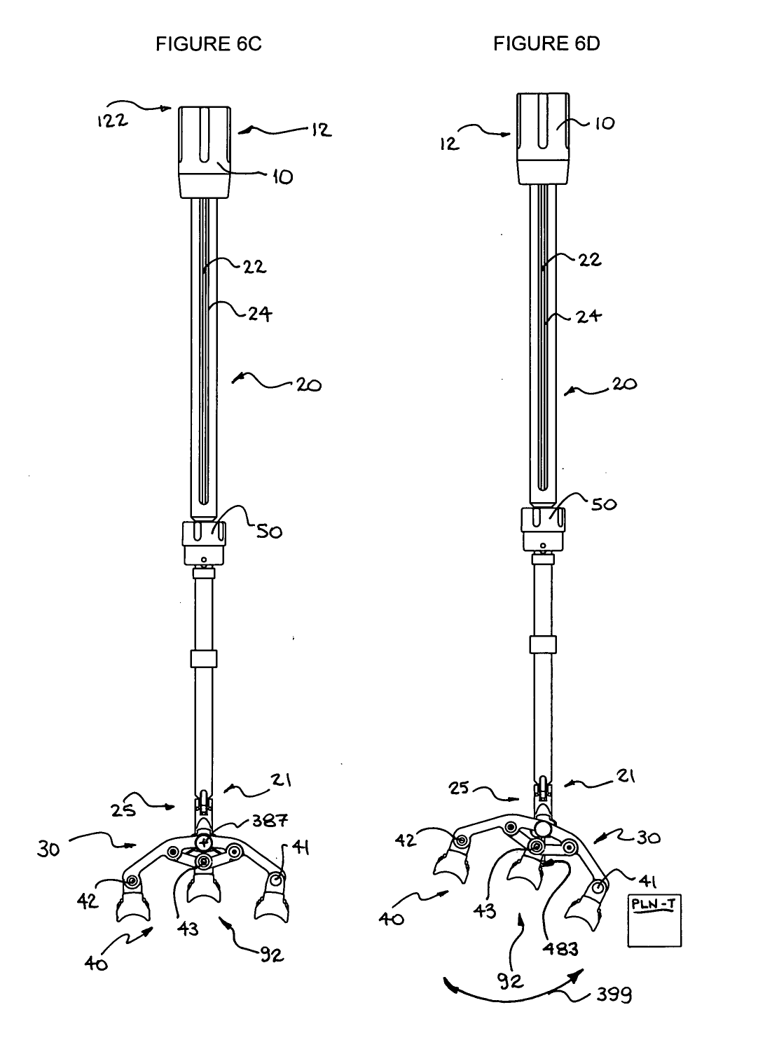

Movable linkage mechanism 30 is comprised of a plurality of movable linkage members. Each linkage member is pivotingly connected or coupled to at least one other linkage member comprising said linkage mechanism 30. With reference to FIGS. 6A-6B, linkage member 36 is pivotingly connected to linkage member 35 through blade mount joint 43, and pivotingly connected to linkage member 38 at linkage joint 386. Linkage member 35 is pivotingly connected to linkage member 37 at linkage joint 375. Linkage members 38 and 37 are pivotingly connected to each other at linkage joint 387.

Generally aligned with blade mount joint 43, linkage mechanism 30 is provided with a socket member 31 configured to receive therewithin ball end 221 of actuating cable 22. As such, linkage mechanism 30 is demountably coupled or connected to actuating cable 22. A locking member, clasp or latch 312 keeps said cable ball end 221 inserted within said socket 31.

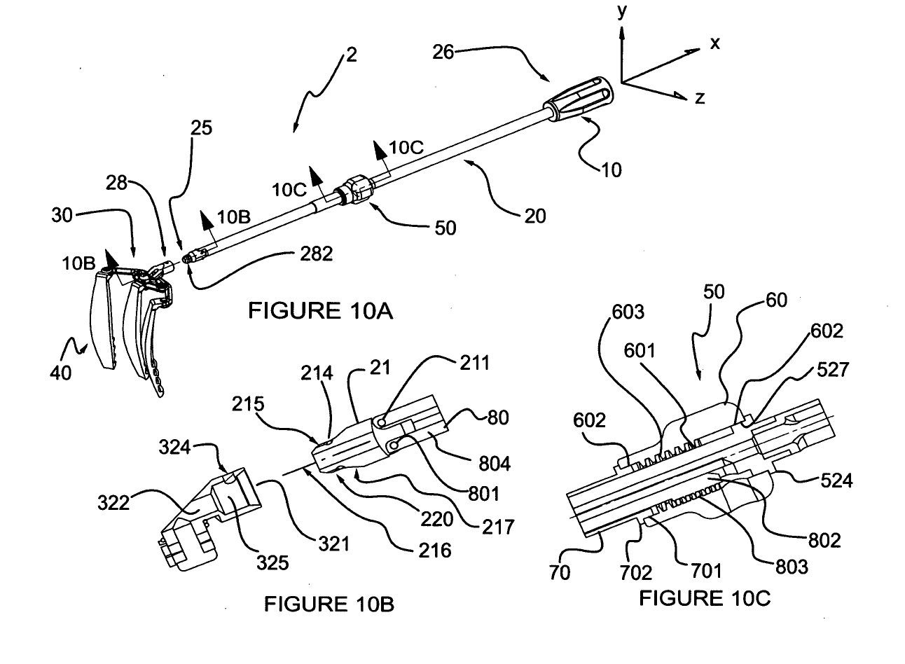

Tissue retractor 1 is described and illustrated in the context of surgery practiced through an intercostal access port IAP, and as such linkage mechanism or assembly 30 is preferably demountably coupled to housing 20 at housing distal end or first housing end 25 through a housing demountable coupling joint or mechanical interface 28. With reference to FIGS. 3-5, a demountable coupling joint 28 in the nature of an opposed tapered surfaces or wedge joint 281. With reference to FIGS. 2 and 10, a demountable coupling joint 28 in the nature of a splined mechanical joint 282. Other types of demountable mechanical joints are also possible such as a bayoneted joint, or a threaded joint, or a spring loaded latch joint. In a heart surgery practiced through a sternotomy approach the necessity for a demountable coupling joint 28 may not be necessary since the access to the anatomic tissue to be retracted may be through a sufficiently large opening (such as a sternotomy incision and retracted ribcage) that tissue retractor 1 does not need to be inserted through a separate stab incision SI.

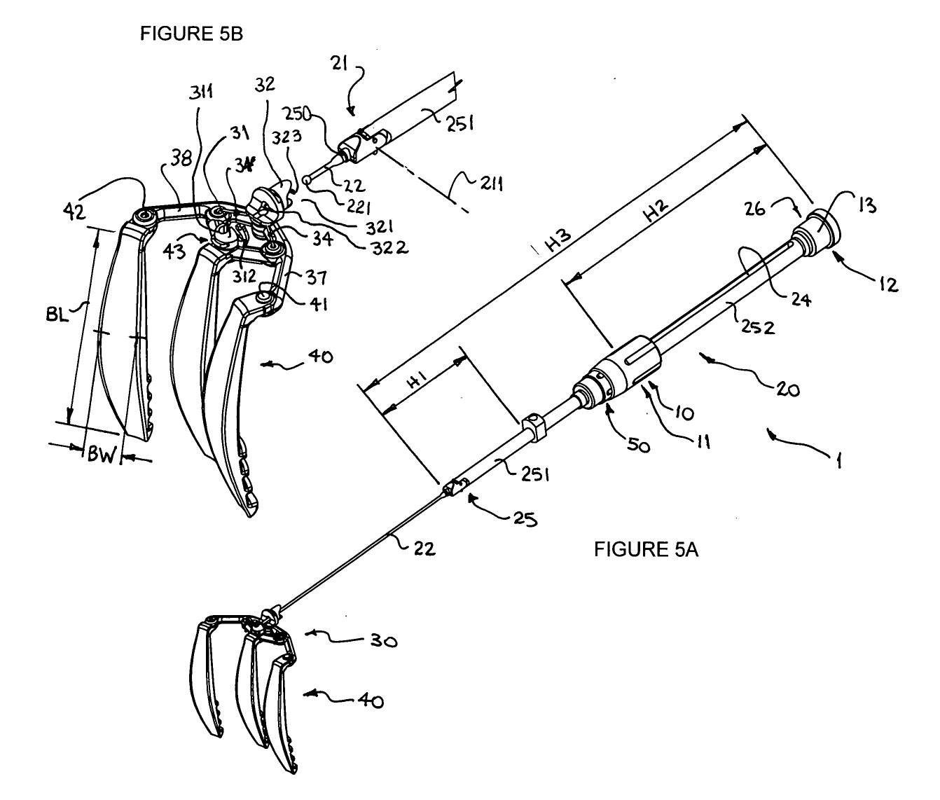

With said linkage mechanism 30 engaged at housing coupling joint 28, a translational movement of cable 22 through housing 20 will entrain a pivoting of the linkage members 35, 36, 37, 38 relative to each other and a simultaneous movement of blades 47, 48, 49 relative to each other. More specifically, retracting cable 22 within said housing 20 will result in mechanical joint 43 being drawn in closer proximity to linkage joint 387 and a spacing apart of blades 47, 48, 49. Conversely, extending cable 22 outwardly for said housing end 25 will result in blades 47, 48, 49 moving closer to each other. As such, linkage assembly 30 is able to articulate in a multitude of different linkage configurations, and consequently able to transmit a multitude of blade spatial geometries or blade spaced apart spatial relationships, relative to said housing 20. As such, tissue retractor 1 may be adapted or adjusted to take on a desired retraction geometry as blades 47, 48, 49 are selectively moved by actuation cable 22 between a closed-blade configuration 91 and an open-blade configuration 92. Linkage mechanism 30 is biased by one or several spring means or members acting between adjacent linkage members in a manner to bias the spacing between blades 47, 48, 49 towards a closed-blade configuration 91, wherein said blades are in close proximity relative to one another. For example, as illustrated in FIG. 5B, a spring member 34 consisting of an elongate spring wire bent about joint 387, and coupled to both linkage members 37, 38 at joints 375, 386 respectively, urges linkage members 37, 38 to pivot towards each other about joint 387 and biases plurality of blades 40 towards their closed-blade configuration 91. Consequently, tension in cable 22 is also maintained when linkage assembly 30 is coupled or connected to housing 20. As such, linkage assembly-to-housing coupling 28 is kept in contact or engagement since cable 22 is kept under tension.

Cable 22 is preferably flexible so as to allow flexing of the exposed cable portion extending beyond housing first end 25. When blades 47, 48, 49 are engaged with a cardiac tissue to be retracted, a flexible cable provides further adaptability by allowing the entire linkage mechanism 30 to articulate relative to linkage joint 387. As such, linkage mechanism 30 may orient itself as an entire assembly relative to housing 20, as a function of the resistance exerted by the tissue being retracted, in any one given blade configuration (i.e. blade closed, blade open, or intermediately therebetween). As such, blades 47, 48, 49 (pivotingly attached to linkage mechanism 30) are free to assume a less traumatic orientation relative to tissue being retracted. This said articulation of the entire linkage mechanism 30 relative to joint 387 is illustrated in comparing FIGS. 6C and 6D (in this case the given blade configuration being open-blade configuration 92). This said articulation of entire linkage mechanism 30 (schematically illustrated by curved arrow 399 in FIG. 6D) is free to occur due to flexibility of cable 22, with blade-to-linkage joints 41, 42, 43 moving within plane PLN-T, while plane PLN-T is held in a fixed orientation relative to housing longitudinal axis 29 at a given setting of actuator 50, at a given orientation of wrist joint 21 relative to housing 20. Alternatively, flexible cable 22 may be replaced by a rigid rod member, and as such, said articulation 399 of entire linkage mechanism 30 within plane PLN-T would be prevented.

Housing 20 is elongate extending in length along a longitudinal axis 29 between a first housing distal end 25 and a second housing proximal end 26. Housing 20 is substantially hollow and configured with a centrally disposed passageway or channel or bore 250 extending from said distal end 25 towards proximal end 26. With reference to FIGS. 4-6D, housing 20 is preferably made from a tubular construction having a cylindrical bore 250, and a cylindrical outer surface 251 over length H1 to facilitate insertion of said housing into stab incision SI formed between two adjacent ribs. Length H1 of housing 20 is sufficiently long to cater for variations in patient anatomy such that when said housing 20 is inserted in said stab incision SI, and said housing 20 is clamped at mounting seat 204 in mechanical joint 962 of instrument positioning arm 96, housing distal end 25 will extend sufficiently beyond the patient's ribcage and into the patient's thoracic cavity TC. A transverse longitudinal slot 24 communicates with said bore 210 over a length H2 of housing 20. Over length H2, housing 20 has a cylindrical external surface 252 interrupted only by slot 24. Slot 24 is configured and sized to slidingly engage with fitting or tongue member 222 of cable 22 when said cable 22 is inserted into said bore 250. Slot 24 also serves as an anti-rotation feature keeping actuating cable 22 from rotating when the latter is translated through said housing 20.

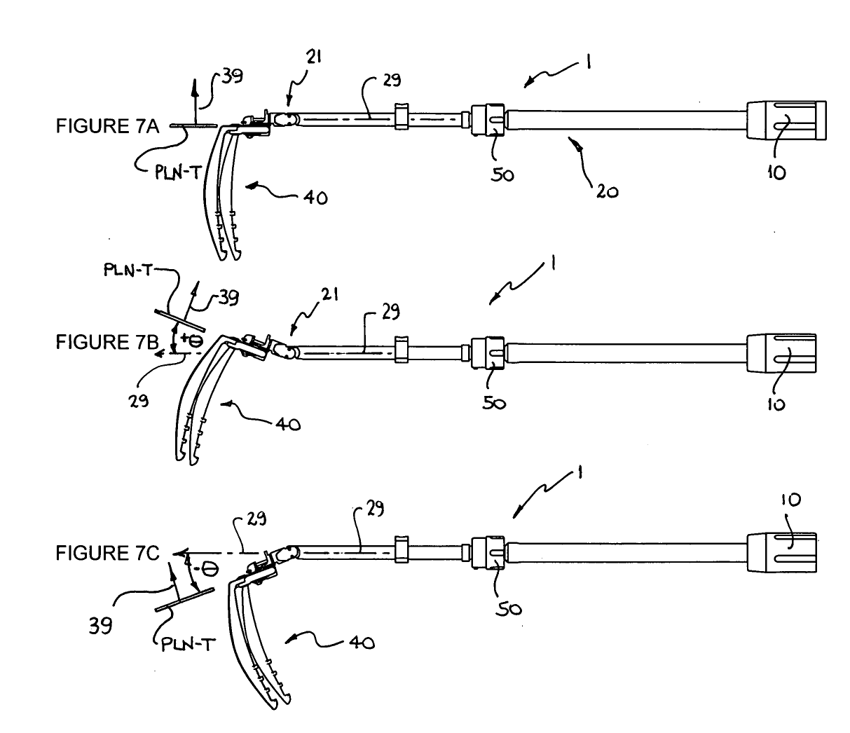

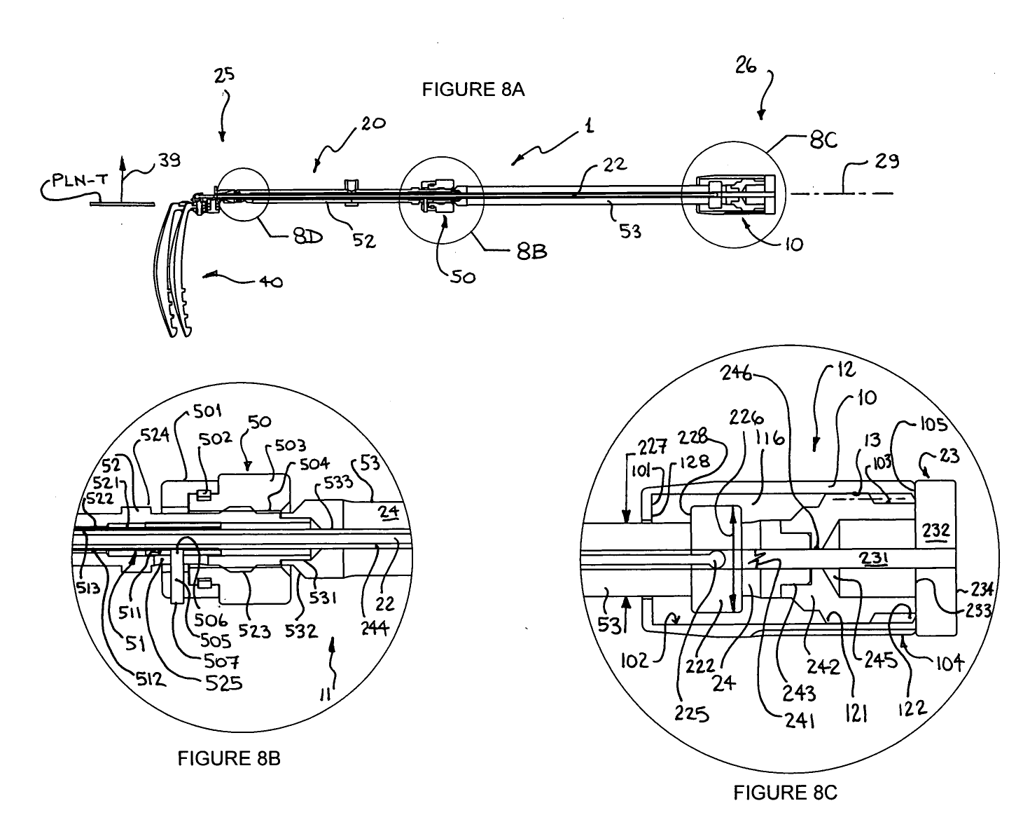

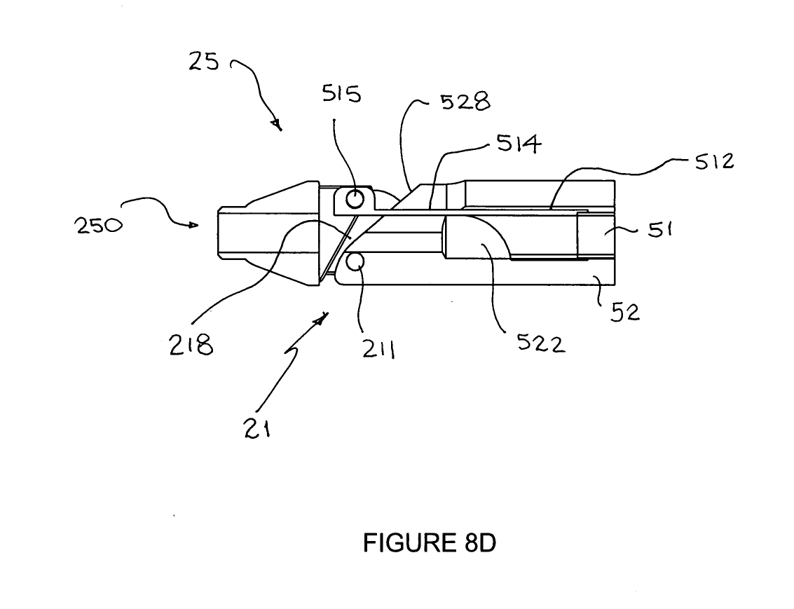

Referring to FIG. 8C, at proximal end 26 of housing 20, a threaded member, fitting or portion 242 is permanently mounted to said housing, preferably through a permanent joint 243. Joint 243 may be a glued joint, a welded joint, a brazed joint, or any other suitable joint that keeps threaded portion 242 permanently connected to said housing during surgical use. Threaded member 242 is configured with an external thread 13 that mates with internal thread 103 on actuator 10. As such, actuator 10 is rotatingly engaged with housing 20 at said threaded interface 103, 13. When an actuation input is applied to actuator 10, in the nature of a rotational input 100, said actuator 10 is movable relative to said housing 20 between a first threaded position 121 (as illustrated in FIG. 6B) and a second threaded portion 122 (as illustrated in FIG. 6C). Said rotational actuation input 100 also results in a movement of actuator 10 along longitudinal axis 29. As well, actuator 10 is slidingly engaged with housing 20 and able to translate or slide relative to said housing over length H2, between a first sliding position 151 and a second sliding position 152 (as illustrated in FIG. 6A).

Length H2 of housing 20 is preferably sized to be between 30 and 70% of housing total length H3, and more preferably to be between 40 and 60% of housing total length H3. As will be described in greater detail below, such housing configuration offers advantages in the deployment of cardiac tissue retractors for valve surgery practiced through an intercostal access port IAP

Actuating member 22 is preferably an elongate flexible cable having a length similar to housing overall length H3. Cable 22 may be of a multi-stranded braided stainless steel construction. At a first distal cable end, cable 22 is configured with an enlarged terminal end, preferably a spherical or ball end 221. Ball end 221 is configured and sized to engage and be demountably coupled to linkage mechanism 30 at socket 31 thereof. As such, actuating cable 22 is coupled to plurality of tissue-engaging blades 40 through linkage mechanism 30 which forms a permanent assembly with said blade plurality 40. At a second proximal cable end, cable 22 is configured with a key or tongue member 222 in a manner to be preferably demountably coupled to actuator 10. Tongue 222 includes two opposed planar surfaces offset by a predetermined depth to allow tongue 222 to be slidingly engaged in housing slot 24. Tongue 222 may be produced by plastic injection by molding over cable protrusion or enlargement 225 to preferably create a permanent mechanical assembly with cable 22. Alternatively, tongue 222 may be produced by other methods to create an appropriately sized key member to slidingly engage slot 24, or may even be a demountable element of cable 22. The width 226 of tongue 222 is larger than the width dimension 227 of housing 20 over housing length H2 so as to create a tongue abutment face or shoulder 228 that is suitably sized to mate and engage with a cooperating abutment shoulder or surface 128 on actuator 10. Tongue width 226 is smaller than the diameter of actuator internal thread 103 so as to allow cable 22 to be inserted in slot 24 and bore 250 and eventually to allow tongue 222 to be insertable within cavity 116 of actuator 10 at the end of cable assembly process. By having cable tongue 222 fittingly engaged within actuator cavity 116, and by virtue of cooperating abutment shoulders 128, 228, actuating cable 22 can be deployed and translate relative to housing 20 when actuator 10 is actuated over the range of actuator positions. As illustrated and described, cable 22 may be demountable from housing 20, mechanism 30, and actuator 10 in order to allow proper cleaning of bore 250 and allow changeover of cables between surgical uses since such flexible braided cables are difficult to clean and re-sterilize. Alternatively, cable 22 may be permanently mounted to actuator 10 through a mechanical joint allowing relative rotation between actuating cable and actuator 10 when said actuator is deployed between first 121 and second 122 threaded positions.

When actuating member or cable 22 is inserted into housing bore 250 and coupled at first end 221 to linkage mechanism socket 31 and at second end 222 coupled to actuator 10, the following configurations are preferred as a function of actuator 10 position relative to housing 20: when actuator 10 is in first sliding position 151, cable 22 is fully extended from housing 20 and blades 47, 48, 49 are in a blade-closed configuration 91; when actuator 10 is in second sliding position 152, linkage mechanism 30 is coupled to housing coupling joint 28 and blades 47, 48, 49 are in a blade-closed configuration 91; when actuator 10 starts to engage a first threaded position 121, blades 47, 48, 49 start to move apart relative to each other away from their blade-closed configuration; when actuator 10 engages a second threaded position 122, blades 47, 48, 49 are in a maximum blade-open configuration 92; when actuator 10 engages a threaded position between threaded position 121 and 122, blades 47, 48, 49 take on an intermediate spaced apart blade relationship between their fully closed and fully open blade configurations. An applied actuation input 100 will deploy, adjust, or adapt the plurality 40 of tissue-contacting blades 47, 48, 49 into a desired spatial arrangement suitable for a surgical procedure. Incremental variations in the actuation input 100 will result in a similar incremental variation in said spatial arrangement of said tissue-engaging blades. As such, a surgeon may apply a predetermined actuation input 100 to said actuator 10 to achieve a desired deployment or adjustment of said tissue-engaging blades 47, 48, 49, said spatial relationship of blades 40 being well suited for the retraction of a specific cardiac tissue, a particular surgical incision, or the surgical exposure of an internal cavity.

Mechanical interface 28 allows linkage assembly 30 to be separated or demountably coupled to housing 20. As such, with blades 40 in first blade-closed configuration 91, linkage assembly 30 and blades 40 connected thereto may be inserted into intercostal access port (labeled IAP) or thoracic port between ribs into thoracic cavity TC. Linkage assembly 30 may then be coupled to cable 22 at socket 31. Retracting cable 22 within housing 20 will draw linkage mechanism 30 into connection with housing coupling 28. Proximal extracorporeal manipulation of substantially tubular housing 20 will place blades 47, 48 and 49 into engagement with atriotomy incision (labeled LAI). Applying a retraction load on housing 20 will cause blade plurality or blade set 40 to apply a retraction to cardiac tissue along LAI thereby obtaining surgical access to a left atrium and a mitral valve (labeled MV) visible therethrough. The relative spacing between blades 47, 48, 49 may be achieved by incrementally and selectively turning actuator knob 10 a desired amount, and as such the resulting atrial opening may be selectively varied by the movement of said cooperating blades.

A housing 20 configuration with features described above is advantageous in surgeries where it is desirable to have an actuation member 22 that is extendible from its housing, for example in valve surgeries practiced through a minimally invasive port access incision IAP, in order to facilitate the coupling of said actuation member 22 with a plurality of tissue engaging blades 40 (and their linkage mechanism 30) that together are too voluminous to be insertable into a thoracic cavity through IAP. More specifically, with the above advantageous housing configuration, an actuation cable 22 of length similar to housing length H3, said cable end 221 may be extended a considerable length (i.e. a cable extension substantially equal to dimension H2) beyond housing end 25. Consequently, while said housing 20 is already inserted in stab incision SI (see FIG. 2), cable end 221 may be extended sufficiently beyond housing end 25 and also out through IAP to permit cable ball end 221 to be inserted in socket 31 of linkage mechanism 30 extracorporeally.

Referring to FIGS. 1, 2, 6A through 7C, the deployment of cardiac tissue retractor 1 will be described in greater detail with reference to a surgical method for practicing a surgical intervention on a mitral valve MV, through a left atrial incision LAI and an intercostal surgical approach. The steps include:
performing an intercostal surgical incision between two adjacent ribs of the patient's ribcage to access the patient's thoracic cavity TC;
inserting blades 971 and 981 of a thoracic retractor 99 into said intercostal incision and deploying said retractor 99 in a manner to engage said blades 971, 981 with patient's ribcage and, if and as required, spreading apart said ribs a desired amount to create an intercostal access port IAP;
exposing the patient's heart HRT as per cardiac surgical procedures practiced through an intercostal surgical approach (i.e. displace lungs, incise pericardium, retract pericardium, mobilize heart within thoracic cavity, etc.);
performing a left atrial or atriotomy incision LAI in the patient's heart HRT, in a manner to obtain a surgical access into the patient's left atrium cavity;
assembling obturator 23 into housing 20, and ensuring obturator tip 235 extends through housing bore 250 beyond housing end 25;
inserting housing 20 and obturator 23 assembly into a separate stab incision SI, located adjacent IAP, in a manner that housing distal end 25 is located within the patient's thoracic cavity TC;
withdrawing obturator 23 from housing 20;
assembling cable end 221 into housing slot 24 while actuator 10 is in descended position 11 or its first sliding position 151, extending cable end 221 past housing first end 25 into TC and extracorporeally out through IAP, and ensuring cable fitting 222 is housed within actuator cavity 116;
coupling ball end 221 to the assembly consisting of linkage mechanism 30 and plurality of tissue-engaging blades 40 at socket 31;
retracting cable 22 through housing 20 (and drawing into thoracic cavity TC plurality of tissue-engaging blades 40) by sliding actuator 10 over housing distance H2 between first sliding position 151 and second sliding position 152;
engaging housing coupling joint 28 between housing 20 and linkage mechanism 30 when actuator 10 begins to rotatingly engage housing threaded portion 13 at a first threaded position 121;
applying a rotational actuation input 100 to actuator 10 to impart a desired spaced apart spatial relationship between blades 47, 48, 49 suitable for inserting tissue-retracting blades 40 into LAI;
extracorporeally rotating housing 20 about its longitudinal axis 29 in a manner that suitably orients the plurality of blades 47, 48, 49 relative to LAI;
proximally and extracorporeally manipulating housing 20 in manner to insert blades 47, 48 and 49 into LAI and placing said blades into engagement with left atrium cardiac tissue to be retracted;
adjusting, as necessary, the relative spacing between blades 47, 48, 49 by incrementally and selectively applying an actuation input 100 to actuator 10;
extracorporeally applying a retraction load to housing 20 in a manner to suitably and sufficiently retract the incised left atrial cardiac tissue a desired amount so as to gain surgical access into the left atrium cavity and to the target mitral valve MV;
securing the position and orientation of tissue retractor 1 (that imparts the above desired retraction load), relative to thoracic retractor 99, by clamping housing 20 at mounting seat 204 to mechanical joint 962 of positioning arm 96;
if and as required during the surgical procedure, changing the angular orientation of blade set 40 relative to housing longitudinal axis 29 by applying an actuation input 500 to actuator 50 to articulate wrist joint 21.

The fine tuning of the relative spacing between blades 47, 48, 49 may be carried out at any time during the above process when linkage mechanism 30 is engaged with housing 20, by incrementally and selectively deploying actuator knob 10 a desired amount. As well, the fine tuning of the angular rotation of blade set 40 relative to wrist joint axis 211 (and angular orientation of blade set 40 relative to housing 20 and more specifically housing longitudinal axis 29) may be carried out at any time during the above process when linkage mechanism 30 is engaged with housing 20, by incrementally and selectively deploying actuator knob 50 a desired amount.

To facilitate fabrication, housing 20 is preferably comprised of a first distal housing member 52 and a second slotted proximal housing member 53, said members being permanently joined at interface 531.

Referring to FIG. 8B, second actuator 50 is comprised of a rotating knob 503 having an outer diameter that is preferably textured or provided with grooves 509 to allow the user to securely apply a sufficiently high moment or actuation input 500 relative to distal housing 52 without slipping. Inner diameter thread 504 in second actuator knob 503 is engaged with the outer diameter thread 523 of the distal housing 52 such that a rotation 500 applied to the second actuator knob 503 causes it to translate along axis 29 of distal housing 52. Said distal housing is provided with an abutment member or shoulder 524 that limits the allowable translation of second actuator knob 503 and coupling member 501 relative to distal housing 52 along axis 29, in the distal direction toward housing first end 25. Proximal housing 53 is provided with a shoulder member 532 that limits the axial movement or translation of second actuator 50 along axis 29 in a direction towards housing second end 26. Proximal housing 53 is configured with a central longitudinal passageway or lumen 244, in open communication with slot 24, to allow cable 22 to be inserted and housed therewithin.

Coupling 501, being engaged with second actuator knob 503 through retaining ring 502, thereby can transmit a corresponding translation along axis 29 to inner translating actuation tube 51. Axial motion of said tube 51 is imparted by knob 503 through transverse pin 505, which is simultaneously engaged with coupling 501 at pin outer extremity 507 and with inner tube proximal end 511 at pin inner extremity 506. Inner tube 51 is guided within a proximal lumen 521 of distal housing 52.

Distal end 512 of inner actuation tube 51 is guided within a distal lumen 522 of distal housing 52. The translation of inner actuation tube 51 resulting from an actuation input 500 to second actuator 50 serves to actuate or articulate wrist joint 21 relative to housing 20. Slot 525 of distal housing 52 prevents rotation of coupling 501 relative to distal housing 52, thus rotation of second actuator knob 503 relative to distal housing 52 results in a translation of inner actuation tube 51 relative to distal housing 52 along axis 29.

Referring to FIG. 8B, inner actuation tube 51 is configured or disposed with a central lumen 513 to allow passage of actuating cable 22 therethrough. As such, a compact housing arrangement results whereby cable 22 is able to freely translate within said lumen 513 and transmit actuation input 100 applied to first actuator 10 to plurality of tissue-engaging blades 40 independently of a second actuation input 500 that may be applied at second actuator 50 to articulate wrist joint 21.

Referring to FIG. 8C, first actuator 10 has an outer diameter 104 that is preferably textured or configured with slots or recesses 109 to allow the user to securely apply a sufficiently high moment or actuation input 100 to rotate actuator knob 10 relative to proximal housing 53 without slipping. First actuator knob 10 is disposed with inner diameter thread 103 over a portion of inner diameter 102, and is engaged with outer diameter thread 13 of proximal housing fitting 242. Proximal housing fitting 242 is configured or disposed with lumen 246 to allow passage of obturator rod 231 of obturator 23. In a temporary assembly of cardiac tissue retractor 1, the obturator rod 231 is installed within lumen 244 through an enlarged and conically tapered guide hole 245 until obturator tip 235 protrudes from distal lumen 212 beyond wrist joint 21. As such, protruding obturator tip 235 facilitates the insertion of distal housing end 25 into a patient's body, and as illustrated, through a stab incision SI into thoracic cavity TC. Obturator 23 is disposed with button 232, preferably welded to obturator rod 231, and having outer face 234 against which a user may apply a force to drive said obturator tip 235 and distal housing 52 into a thoracic cavity TC. Outer face 234 is configured and sized with a sufficiently large surface area to minimize pressure to users hand during insertion into thoracic cavity TC. A force applied to obturator is transmitted to retractor housing 20 through contact between obturator inner face 233 and first actuator outer face 105. Referring to FIG. 8C, only a proximal short portion of obturator 23 extending between break line 241 and outer face 234 is illustrated engaged with tissue retractor 1.

Once retractor housing 20 is inserted into stab incision SI, obturator 23 having fulfilled its purpose of facilitating the insertion of said housing into the thoracic cavity, can be withdrawn from housing 20 by pulling on obturator button 232, thereby liberating lumen 244 (and housing bore 250) for subsequent insertion of cable 22.

Installation of blades 40 on cable 22 proceeds by first introducing distal end 221 of cable 22 into proximal end 321 of linkage coupling member 32 and pushing it through opening 322 until cable ball end 221 can be inserted into top side 311 of socket 31. At this point, clasp or latch member 312 can be rotated over cable portion engaged in said socket to engage said cable with linkage assembly 30. Retraction of said cable through housing 20 will in a first instance bring into contact mechanical joint 28 (while plurality of blades 40 remain in a blade-closed configuration) and once said linkage mechanism 30 is in contact with housing 20 at said joint 28, further retraction of said cable 22 within housing 20 will progressively spread apart blades 40 between blade-closed configuration 91 and blade-open configuration 92 through the actuation of first actuator 10.

Referring to FIG. 8D, a first embodiment of wrist joint 21, distal tube 512 of inner actuation tube 51 is shaped to have flexible member 514 that engages wrist joint 21 at pinned interface 515. Translation of inner actuation tube 51 relative to distal housing 52 through the application of actuation input 500 at second actuator 50 causes wrist joint 21 to rotate about pivot axis 211, thus effecting a change in orientation of retractor blades 47, 48, 49 relative to housing longitudinal axis 29, and about wrist joint pivot axis 211. The range of angular orientation is only limited in a first direction by the contact between rear face 218 of wrist pivot 21 and front face 528 of distal housing 52, and in a second direction by contact between coupling 510 of knob 50 and shoulder 524 of distal housing 52. Application of second actuation input 500 to second actuator 50 results in housing first end 25 rotating or bending relative to housing axis 29 (and relative to housing second end 26) about pivot axis 211.

With reference to FIGS. 7A-7C, actuating actuator 50 and pivoting wrist joint 21 will result in a change in orientation of PLN-T (and a change in direction of vector 39) relative to housing axis 29, from a perpendicular relationship to long axis 29 as illustrated in FIG. 7A when said actuator knob 50 is for instance in its home position, to an angle >90 degrees when actuator 50 is rotated in a first direction relative to housing 20 as illustrated in FIG. 7B, and to an angle <90 degrees when the actuator 50 is rotated in an opposite second direction relative to housing 20. As such, PLN-T can change its orientation between +/−Θ relative to a plane parallel to both axis 29 and 211. Consequently, blades 40 that are connected to linkage mechanism 30 will also change their angular orientation relative to pivot axis 211 and axis 29 as said PLN-T undergoes the above change in orientation.

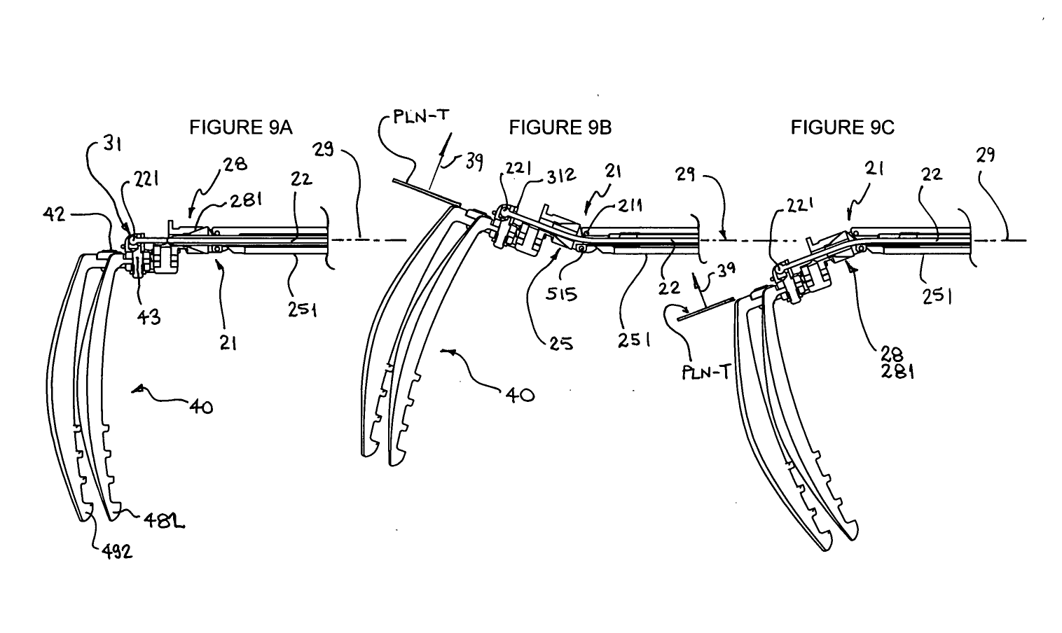

Referring to FIGS. 9A-9C, the flexing of cable 22 is visible as wrist joint is actuated and PLN-T is reoriented. When said second actuator 50 is actuated, the angular orientation of said plane PLN-T relative to said housing longitudinal axis 29 is changeable, said change in angular orientation being proportional to the degree of pivoting at said wrist pivot joint 21 which imparts a corresponding angular displacement of said plane PLN-T about said wrist joint pivot axis 211.

In a first embodiment, cardiac tissue retractor 1 is provided with a demountable coupling joint 281 which permits only two coupling arrangements between linkage mechanism 30 and housing 20. When first housing end 25 and second housing end 26 are aligned with longitudinal axis 29, said joint 281 allows two angular orientations of PLN-T (vector 39 as illustrated in FIG. 3 with blades 40 extending downwards, or with vector 39 in opposite direction to as illustrated in FIG. 3 with blades extending upwards). In both of these angular orientations, vector 39 is perpendicular to axis 211 and housing axis 29. When wrist joint 21 is articulated by actuating actuator 50, housing first end 25 bends relative to housing second end 26 about pivot axis 211, and vector 39 changes its angular orientation relative to housing axis 29 by rotating about pivot axis 211. In both these coupling arrangements, PLN-T rotates relative to PLN-W when wrist joint 21 pivots, but PLN-T remains perpendicular to PLN-W. As actuator 50 is actuated, PLN-T will change its angular orientation relative to axis 29, said change in angular orientation being proportional to the amount of actuation input 500 applied at actuator 50.

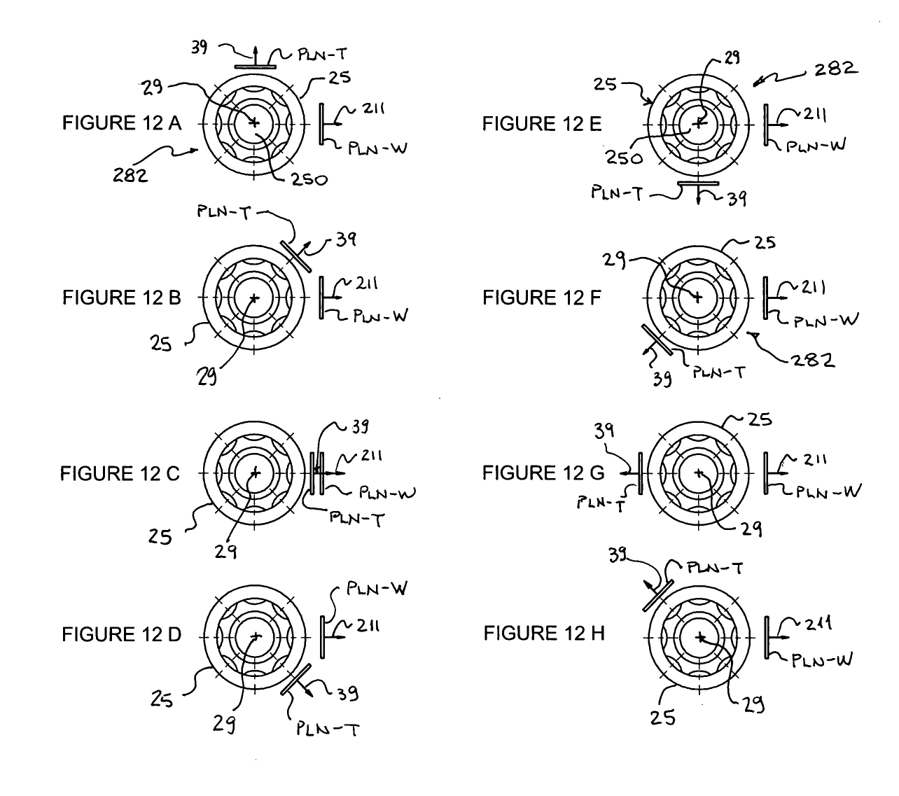

Referring to FIGS. 10A-10C, a second embodiment of cardiac tissue retractor 2 is described offering a variety of coupling arrangements through demountable coupling joint 282 between linkage mechanism 30 and housing 20. When first housing end 25 and second housing end 26 are aligned with longitudinal axis 29, said joint 282 allows eight angular orientations of PLN-T relative to PLN-W. These coupling variations are illustrated in FIGS. 12A-12H. When wrist joint 21 is articulated by actuating actuator 50, housing first end 25 bends relative to housing second end 26 about pivot axis 211, and vector 39 changes its angular orientation relative to housing axis 29 by rotating about pivot axis 211. In coupling arrangements FIGS. 12A and 12 E, cardiac tissue retractor 2 behaves as does cardiac tissue retractor 1. In coupling arrangements 12B, 12D, 12F, and 12H, PLN-T varies its angular relationship relative to PLN-W when wrist joint 21 pivots. In coupling arrangements 12 C and 12G, PLN-T remains parallel to PLN-W when wrist joint 21 pivots. Other demountable coupling arrangements offering more or less coupling arrangements are also possible without departing from the spirit of the invention.

of second actuator 50 has knob 60 with proximal inner diameter 602 guided on outer diameter 527 of distal housing 52, and distal inner diameter 604 guided on outer diameter 701 of fitting 70. Fitting 70 is fixed relative to distal housing 52 preferably by welding. Knob 60 can thus rotate freely and is substantially limited in its axial movement by virtue of being trapped between shoulder 524 of housing 52 and shoulder 702 of fitting 70. Actuator rod 80 has thread sector 803 at proximal end 802 that is engaged with inner thread 601 of second actuator knob 60 such that a rotation of actuator knob 60 causes actuator rod 80 to translate in a direction substantially parallel to axis 526 of distal housing 52. The translating rod 80 causes pinned joint 801 to orbit about wrist joint axis 211, thus changing the orientation of wrist joint 21 relative to axis 526.

Referring to FIG. 10B, a second embodiment of mechanical interface 28 consists of multiples of slot 215 dispositioned around wrist pivot 21 and arranged to permit multiple choices for the angular orientation of linkage coupling 32 relative to pivot axis 211. Pin 324 in linkage coupling 32 engages slot 215 when wrist joint 21 is introduced into socket 325 of linkage coupling 32 and thus limits rotation of linkage coupling 32 about axis 216 of wrist pivot 21 once engaged. Wrist joint 21 is also disposed with multiple sockets 214 around its circumference, each socket coincident with proximal end 217 of each slot 215 such that an axial load oriented distally in a direction substantially parallel to lumen axis 216 of wrist pivot 21 causes pin 324 to positively engage with socket 214 and prevent disengagement of retractor linkage 30 from wrist pivot 21.

Cleaning port 283 is provided in housing 20 to allow flushing and cleaning of internal bore 250 and passages and lumens contained within housing 20.

As illustrated in FIG. 8A, cable 22 is co-linear with housing longitudinal axis 29 and in the same plane as wrist axis 211. This makes for a compact arrangement resulting in a housing with small cross sectional area to advantageously minimize size of stab incision SI.

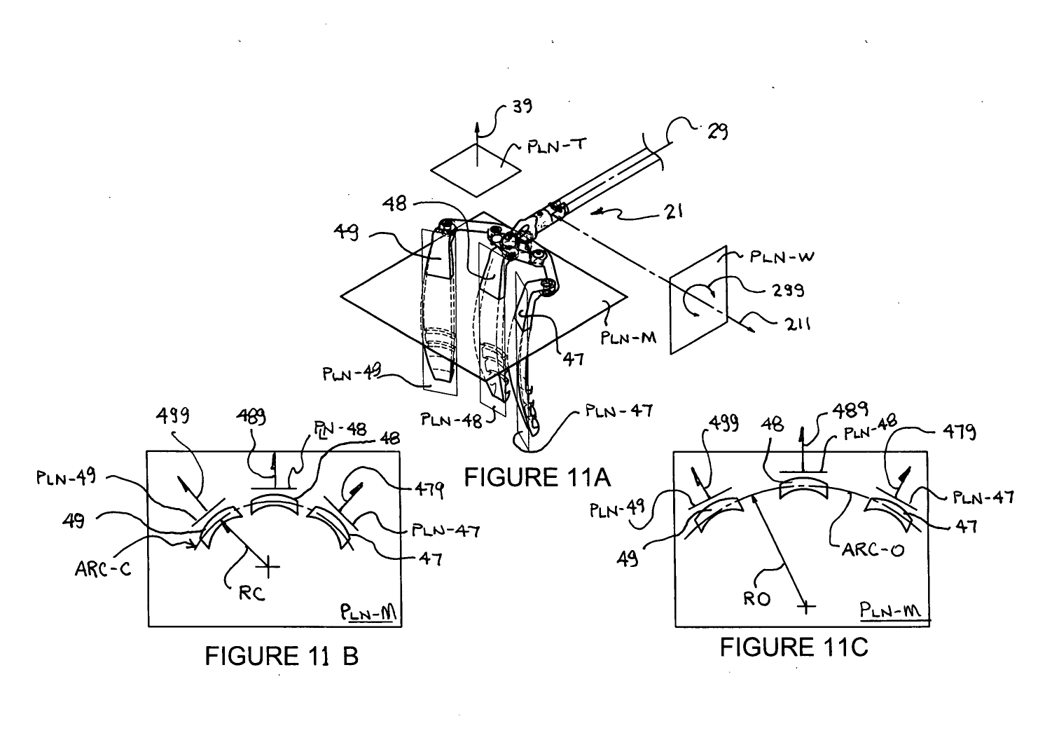

Rotating actuator 50 will impart an angular rotation of blades 47, 48, 49 (shown schematically with arcuate arrow 299 in FIG. 11A about the wrist axis 211.

Also with reference to FIG. 11A-11C, each of blades 47, 48, 49 cooperate to impart a retraction load to a cardiac tissue generally along a retraction plane PLN-47, PLN-48, PLN-49, respectively. Said planes are also defined by a retraction vector 479, 489, 499. As illustrated in FIG. 11A, said blades preferably extend substantially perpendicular to plane PLN-T through the blade-to-linkage mounts 42, 41, 43, and as such retraction planes PLN-47, PLN-48, PLN-49 are illustrated perpendicular to plane PLN-T and plane PLN-M which is offset parallel to PLN-T and cuts through the mid-span height BL of said blades. The angle between each of the respective retraction plane vectors 479, 489, 499 and housing longitudinal axis 29 may be varied by the application of an actuation input 500 to actuator 50. As well, said actuation input 500 will impart an angular rotation of the retraction planes PLN-T, PLN-47, PLN-48, PLN-49 about the wrist pivot axis 211.

Referring to FIG. 11B, blades 47, 48, 49 are illustrated in a first closed-blade configuration. An arc of retraction may be defined by ARC-C, having a radius of retraction RC. When actuator 10 is actuated, blades 47, 48, 49 move relative to one another to assume a spaced apart blade-open configuration, whereby the resulting arc of retraction ARC-O is defined by a larger radius of retraction RO. As well, the span of retraction (arcuate distance between blade 47 and 49 along ARC-C) when said blades are in closed-blade configuration (FIG. 11B) is smaller than the span of retraction when said blades are in open-blade configuration (FIG. 11C).

When first actuator 10 is actuated, said blade 47, 48, 49 move between said closed-blade and said open-blade configuration, and whereby when second actuator 50 is actuated, direction of vectors 479, 489, 499 defining their respective retraction planes changes relative to housing longitudinal axis 29, said change in vector direction being proportional to the degree of pivoting at wrist pivot joint 21 that occurs as a function of actuation input 500 applied at actuator 50. Said degree of pivoting at wrist joint 21 about pivot axis 211 imparts a corresponding angular displacement of said vectors about said wrist joint pivot axis.

1. A surgical retractor 1 for retracting a target anatomic tissue of a patient body during surgery, said surgical retractor comprising:
A plurality of tissue-retracting blades, said blades being configured and sized to retract a target anatomic tissue,
a movable linkage arrangement, said linkage arrangement comprising an array of cooperating linkage members operatively coupled to each other, each of said tissue-retracting blades being connected to at least one of said linkage members at a blade-to-linkage joint,
a retractor housing, said retractor housing being generally elongate and extending between a first housing end and a second housing end along a housing longitudinal axis, said linkage arrangement being coupled to said retractor housing at said first housing end, said retractor housing including a pivoting wrist joint, said wrist joint being configured adjacent to said housing first end, said wrist joint capable of bending said housing first end relative to said housing second end about a pivot axis, said pivot axis being substantially perpendicular to said housing longitudinal axis,
a first actuator, said first actuator for actuating the movement of said linkage arrangement, said first actuator coupled to said retractor housing and also to said linkage arrangement via a movable actuation member, whereby when an actuation input is applied to said first actuator, said tissue-retracting blades are movable between a closed-blade configuration wherein said blades are in proximity to one another and an open-blade configuration wherein said blades are in a spaced apart spatial relationship relative to one another,
a second actuator, said second actuator for actuating the pivoting movement of said wrist joint, said second actuator coupled to said retractor housing and to said pivoting wrist joint via a movable actuation member, whereby when an actuation input is applied to said second actuator, said tissue-retracting blades are pivotable about said pivot axis through the pivoting of said wrist joint, said pivoting displacement of tissue-retracting blades being separate and independent to the blade spatial configuration imparted to said tissue-retracting blades by actuating said first actuator.

2. A tissue retractor according to claim 1, wherein said target anatomic tissue is a cardiac tissue, said plurality of tissue-retracting blades comprises three cooperating tissue-retracting blades suitably configured and sized to retract said cardiac tissue, said blade-to-linkage joints defining a first plane, said three cooperating tissue-retracting blades being elongate and extending away from their respective blade-to-linkage joint in a direction generally perpendicular to said first plane, whereby when said first actuator is actuated, said blade-to-linkage joints move relative to one another within said first plane as said tissue retracting blades move between said closed-blade and said open-blade configuration, and whereby when said second actuator is actuated, the angular orientation of said first plane relative to said housing longitudinal axis is changeable, said change in angular orientation being proportional to the degree of pivoting at said wrist pivot joint which imparts a corresponding angular displacement of said first plane about said wrist joint pivot axis.

3. A tissue retractor according to claim 1, wherein said target anatomic tissue is a cardiac tissue, said plurality of tissue-retracting blades comprises first, second and third tissue-retracting blades suitably configured and sized to retract said cardiac tissue, each of said tissue-retracting blades being elongate and extending generally along a first, second, and third tissue retracting plane, respectively, said tissue retracting planes being defined respectively by a first, second and third vector direction, whereby when said first actuator is actuated, said first, second and third blades move between said closed-blade and said open-blade configuration, and whereby when said second actuator is actuated, said vector direction of retraction plane relative to said housing longitudinal axis are changeable, said change in vector direction being proportional to the degree of pivoting at said wrist pivot joint which imparts a corresponding angular displacement of said vectors about said wrist joint pivot axis.
